SEQUENCE LISTING (22 sequences)

SEQ ID NO: 1 - protein, 180 aa, synthetic construct
  REGION 1..180: note = FCeRI1 ECD
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQ

SEQ ID NO: 2 - protein, 215 aa, synthetic construct
  REGION 1..215: note = Modified Fc
SHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 30 aa, synthetic construct
  REGION 1..30: note = IgD hinge variant
RNTGRGGEEKKGSKEKEEQEERETKTPECP

SEQ ID NO: 4 - protein, 49 aa, synthetic construct
  REGION 1..49: note = IgD hinge variant
AQPQAEGSLAKATTAPATTRNTGRGGEEKKGSKEKEEQEERETKTPECP

SEQ ID NO: 5 - DNA, 540 bp, synthetic construct
  misc_feature 1..540: note = nucleotides sequence of FCeRI1 ECD
  source 1..540: mol_type = other DNA
gtgccccagaagcccaaggtgagcctgaaccctccctggaacagaatcttcaagggcgagaacgtgaccctgacctgcaacggcaacaacttcttcgaggtgagcagcaccaagtggttccacaatggcagcctgagcgaggagaccaacagctccctgaacatcgtgaacgccaagttcgaggacagcggcgagtacaagtgccagcaccagcaggtgaacgagagcgagcccgtgtacctggaggtgttcagcgactggctgctgctgcaggccagcgccgaggtggtgatggagggccagcccctgttcctgagatgccacggctggagaaactgggacgtgtacaaggtgatctactacaaggatggcgaggccctgaagtactggtacgagaaccacaacatctccatcaccaacgccaccgtggaggacagcggcacctactactgcacaggcaaggtgtggcagctggactacgagagcgagcccctgaacatcaccgtgatcaaggctcccagagagaagtactggctgcag

SEQ ID NO: 6 - DNA, 561 bp, synthetic construct
  misc_feature 1..561: note = nucleotides sequence of modified Fc
  source 1..561: mol_type = other DNA
tgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 7 - DNA, 174 bp, synthetic construct
  misc_feature 1..174: note = nucleotides sequence of IgD hinge variant
  source 1..174: mol_type = other DNA
aggaacaccggcagaggaggcgaggaaaagaaaggaagcaaggagaaggaggagcaggaggaaagagaaaccaagacccccgagtgccccagccacacccagcccctgggcgtgttcctgttcccccccaagcccaaggacaccctgatgatcagcagaacccccgaggtgacc

SEQ ID NO: 8 - DNA, 231 bp, synthetic construct
  misc_feature 1..231: note = nucleotides sequence of IgD hinge variant
  source 1..231: mol_type = other DNA
gcccagccccaggccgagggcagcctggctaaggccaccacagctcccgccaccaccaggaacaccggcagaggaggcgaggaaaagaaaggaagcaaggagaaggaggagcaggaggaaagagaaaccaagacccccgagtgccccagccacacccagcccctgggcgtgttcctgttcccccccaagcccaaggacaccctgatgatcagcagaacccccgaggtgacc

SEQ ID NO: 9 - protein, 25 aa, synthetic construct
  REGION 1..25: note = signal peptide
MDAMLRGLCCVLLLCGAVFVSPSHA

SEQ ID NO: 10 - DNA, 75 bp, synthetic construct
  misc_feature 1..75: note = nucleotides sequence of signal peptide
  source 1..75: mol_type = other DNA
atggacgccatgctgagaggcctgtgctgtgtgctgctgctgtgcggcgccgtgttcgtgtcccctagccacgcc

SEQ ID NO: 11 - protein, 450 aa, synthetic construct
  REGION 1..450: note = FceRIa ECD-hinge-Fc2
MDAMLRGLCCVLLLCGAVFVSPSHAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQRNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - DNA, 1350 bp, synthetic construct
  misc_feature 1..1350: note = nucleotides sequence of FceRIa ECD-hinge-Fc2
  source 1..1350: mol_type = other DNA
atggacgccatgctgagaggcctgtgctgtgtgctgctgctgtgcggcgccgtgttcgtgtcccctagccacgccgtgccccagaagcccaaggtgagcctgaaccctccctggaacagaatcttcaagggcgagaacgtgaccctgacctgcaacggcaacaacttcttcgaggtgagcagcaccaagtggttccacaatggcagcctgagcgaggagaccaacagctccctgaacatcgtgaacgccaagttcgaggacagcggcgagtacaagtgccagcaccagcaggtgaacgagagcgagcccgtgtacctggaggtgttcagcgactggctgctgctgcaggccagcgccgaggtggtgatggagggccagcccctgttcctgagatgccacggctggagaaactgggacgtgtacaaggtgatctactacaaggatggcgaggccctgaagtactggtacgagaaccacaacatctccatcaccaacgccaccgtggaggacagcggcacctactactgcacaggcaaggtgtggcagctggactacgagagcgagcccctgaacatcaccgtgatcaaggctcccagagagaagtactggctgcagaggaacaccggcagaggaggcgaggaaaagaaaggaagcaaggagaaggaggagcaggaggaaagagaaaccaagacccccgagtgccccagccacacccagcccctgggcgtgttcctgttcccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 13 - protein, 469 aa, synthetic construct
  REGION 1..469: note = FceRIa ECD-hinge-Fc3
MDAMLRGLCCVLLLCGAVFVSPSHAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQAQPQAEGSLAKATTAPATTRNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - DNA, 1407 bp, synthetic construct
  misc_feature 1..1407: note = nucleotides sequence of FceRIa ECD-hinge-Fc3
  source 1..1407: mol_type = other DNA
atggacgccatgctgagaggcctgtgctgtgtgctgctgctgtgcggcgccgtgttcgtgtcccctagccacgccgtgccccagaagcccaaggtgagcctgaaccctccctggaacagaatcttcaagggcgagaacgtgaccctgacctgcaacggcaacaacttcttcgaggtgagcagcaccaagtggttccacaatggcagcctgagcgaggagaccaacagctccctgaacatcgtgaacgccaagttcgaggacagcggcgagtacaagtgccagcaccagcaggtgaacgagagcgagcccgtgtacctggaggtgttcagcgactggctgctgctgcaggccagcgccgaggtggtgatggagggccagcccctgttcctgagatgccacggctggagaaactgggacgtgtacaaggtgatctactacaaggatggcgaggccctgaagtactggtacgagaaccacaacatctccatcaccaacgccaccgtggaggacagcggcacctactactgcacaggcaaggtgtggcagctggactacgagagcgagcccctgaacatcaccgtgatcaaggctcccagagagaagtactggctgcaggcccagccccaggccgagggcagcctggctaaggccaccacagctcccgccaccaccaggaacaccggcagaggaggcgaggaaaagaaaggaagcaaggagaaggaggagcaggaggaaagagaaaccaagacccccgagtgccccagccacacccagcccctgggcgtgttcctgttcccccccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 15 - protein, 406 aa, synthetic construct
  REGION 1..406: note = human a-2,6 sialic acid transferase
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 16 - DNA, 1218 bp, synthetic construct
  misc_feature 1..1218: note = nucleotides sequence of human a-2,6 sialic acid transferase
  source 1..1218: mol_type = other DNA
atgatccacaccaacctgaagaagaagttcagctgctgcgtgctggtgttcctgctgttcgccgtgatctgcgtgtggaaggagaagaagaaaggcagctactacgacagcttcaagctgcagaccaaggagttccaggtgctgaagagcctgggcaagctggccatgggcagcgacagccagagcgtgtccagctcctccacccaggatccccacagaggcagacagaccctgggcagcctgagaggcctggccaaggccaagcccgaggccagcttccaggtgtggaacaaggacagcagcagcaagaacctgatccccagactgcagaagatctggaagaactacctgagcatgaacaagtacaaggtgagctacaaaggacccggacccggcatcaagttcagcgccgaggccctgaggtgccacctgagagaccacgtgaacgtgagcatggtggaagtgaccgacttccccttcaacaccagcgagtgggaaggctacctgcccaaggagagcatcaggaccaaggctggcccctggggcagatgcgccgtggtgagcagcgctggcagcctgaagagctcccagctgggcagagagatcgacgaccacgatgccgtgctgaggttcaatggcgctcccaccgccaacttccagcaggacgtgggcaccaagaccacaatccggctgatgaacagccagctggtgacaaccgagaagcggttcctgaaggacagcctgtacaacgagggcatcctgatcgtgtgggatcccagcgtgtaccacagcgacatccccaagtggtaccagaatcccgactacaacttcttcaacaactacaagacctatagaaagctgcaccccaaccagcccttctacatcctgaagccccagatgccctgggagctgtgggacatcctgcaggagatcagccctgaagagatccagcccaaccctccctccagcggcatgctgggcattatcatcatgatgaccctgtgcgaccaggtggacatctacgagttcctgcccagcaagagaaagaccgacgtgtgctactactatcagaagttcttcgacagcgcctgcaccatgggcgcctaccaccccctgctgtacgagaagaacctggtgaagcacctgaaccagggcaccgacgaggacatctacctgctgggcaaagccaccctgcccggcttcagaaccatccactgc

SEQ ID NO: 17 - protein, 30 aa, synthetic construct
  REGION 1..30: note = IgD hinge variant
  VARIANT 12: note = Xaa is Lys or Gly
  VARIANT 13: note = Xaa is Glu, Gly, or Ser
RNTGRGGEEKKXXKEKEEQEERETKTPECP

SEQ ID NO: 18 - protein, 49 aa, synthetic construct
  REGION 1..49: note = IgD hinge variant
  VARIANT 31: note = Xaa is Lys or Gly
  VARIANT 32: note = Xaa is Glu, Gly, or Ser
AQPQAEGSLAKATTAPATTRNTGRGGEEKKXXKEKEEQEERETKTPECP

SEQ ID NO: 19 - protein, 30 aa, synthetic construct
  REGION 1..30: note = IgD hinge
RNTGRGGEEKKKEKEKEEQEERETKTPECP

SEQ ID NO: 20 - protein, 425 aa, synthetic construct
  REGION 1..425: note = FceRIa ECD-hinge-Fc1
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 425 aa, synthetic construct
  REGION 1..425: note = FceRIa ECD-hinge-Fc2
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQRNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 444 aa, synthetic construct
  REGION 1..444: note = FceRIa ECD-hinge-Fc3
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQAQPQAEGSLAKATTAPATTRNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0062457 A1
(43) Pub. Date: Mar. 5, 2026

(54) EXTRACELLULAR DOMAIN OF ALPHA SUBUNIT OF IGE FC RECEPTOR, PHARMACEUTICAL COMPOSITION COMPRISING SAME AND METHOD FOR PRODUCING SAME

(71) Applicant: GI Innovation, Inc., Seoul (KR)

(72) Inventors: Young Chul Sung, Seoul (KR); Zungyoon Yang, Incheon (KR); Myung Ho Jang, Seoul (KR)

(73) Assignee: GI Innovation, Inc., Seoul (KR)

(21) Appl. No.: 19/294,680

(22) Filed: Aug. 8, 2025

Related U.S. Application Data
(62) Division of application No. 16/958,861, now Pat. No. 12,410,233.

(30) Foreign Application Priority Data
Jan. 8, 2018 (KR) 10-2018-0002248

(51) Int. Cl. C07K 14/735; A23L 33/18; A61K 38/00; A61P 37/08

(52) U.S. Cl. CPC C07K 14/70535; A23L 33/18; A61P 37/08; A61K 38/00

(57) ABSTRACT

Disclosed is a polypeptide dimeric protein containing two monomers, each of which contains an extracellular domain (FcεRIa-ECD) of an alpha subunit of an IgE Fc receptor. The dimeric protein has advantages that an excellent binding ability to IgE is exhibited as compared with a conventional therapeutic agent containing an anti-IgE antibody, and less other side effects are exhibited due to lack of ADCC and CDC functions. Thus, the dimeric protein can be applied to a medical product for treating or preventing an IgE-mediated allergic disease.

### CROSS REFERENCE TO RELATED APPLICATIONS

This application is a Divisional of U.S. application Ser. No. 16/958,861 filed Jun. 29, 2020, which is a National Stage of International Application No. PCT/KR2019/000274 filed Jan. 8, 2019, claiming priority based on Korean Patent Application No. 10-2018-0002248 filed Jan. 8, 2018.

### INCORPORATION BY REFERENCE OF SEQUENCE LISTING

The instant application contains a Sequence Listing which has been filed electronically in xml format and is hereby incorporated by reference in its entirety. Said xml copy, created on Aug. 8, 2025, is named Q312161_SEQ_LIST.xml and is 38,826 bytes in size.

### TECHNICAL FIELD

The present invention relates to modified IgE Fc receptors and uses thereof.

### BACKGROUND ART

Allergic diseases, such as allergic rhinitis, atopic dermatitis, and food allergy, including asthma are spreading at a high rate in industrialized and westernized modern societies, and development of anaphylaxis, a severe allergic disease, is also increasing. These chronic immune diseases severely impair individuals' quality of life and socioeconomic costs are soaring accordingly. Thus, there is a desperate need for measures to overcome such diseases.

Most allergic diseases are caused by an excessive immune response of immunoglobulin E (IgE). IgE is an antibody that is present in serum at a very low concentration under a normal condition. IgE is also usually produced by innocuous antigens. There is a case where the number of IgE is increased without any particular stimulus. Such a case may lead to allergic diseases. The abnormally increased number of IgE can bind to high-affinity IgE Fc receptors (FcεRIs) which are expressed on the surface of mast cells, basophils, and the like. Such binding causes mast cells or basophils to release chemical mediators such as histamine, leukotriene, prostaglandin, bradykinin, and platelet-activating factors. Release of these chemical mediators results in allergic symptoms. In particular, allergic diseases may exhibit worsened symptoms due to the binding between IgE and FcεRI. FcεRI-expressing cells are known to increase in allergic patients.

Currently, various methods, such as allergen avoidance, administration of anti-allergic drugs, modulation of IgE synthesis in the body, and development of anti-IgE antibodies, have been proposed to treat allergic diseases. However, therapeutic methods known so far have many drawbacks, such as inability to cure an underlying cause of allergy, insufficient drug efficacy, and occurrence of serious side effects.

In addition, immunoglobulin compositions capable of binding to IgE and FcγRIIb with high affinity and inhibiting cells expressing IgE have been studied (KR10-1783272B1). Such compositions have been reported to be useful for treating IgE-mediated disorders including allergy and asthma. In addition, omalizumab (trade name: XOLAIR®), which targets an Fc portion of an IgE antibody, has been developed and used as a therapeutic agent for intractable severe asthma and intractable urticaria.

However, a high-dose administration of omalizumab to maintain therapeutic effects leads to a high cost burden, and side effects such as angioedema and anaphylactic reaction (The Journal of Clinical Investigation Volume 99, Number 5, March 1997, 915-925). Besides, from the post-marketing results, allergic granulomatous vasculitis and idiopathic severe thrombocytopenia have been reported. Accordingly, there is a growing need for development of therapeutic agents capable of effectively treating allergic diseases without side effects.

### Technical Problem

An object of the present invention is to provide a polypeptide dimeric protein for treating an IgE-mediated allergic disease. Another object of the present invention is to provide a nucleic acid molecule encoding the protein, an expression vector containing the nucleic acid molecule, and a host cell containing the expression vector. Yet another object of the present invention is to provide a method for preparing the polypeptide dimer.

### Solution to Problem

In order to achieve the above objects, there is provided a polypeptide dimer, comprising two monomers, each of which contains an extracellular domain of an alpha subunit of an IgE Fc receptor. The monomer contains a modified Fc region, and the modified Fc region and the extracellular domain of the alpha subunit of the IgE Fc receptor are linked via a hinge of an IgD antibody. In another aspect, there is provided a pharmaceutical composition for treating or preventing an allergic disease, comprising the polypeptide dimer as an active ingredient.

### Advantageous Effects of Invention

The polypeptide dimeric protein according to the present invention not only has excellent safety and persistence in the body as compared with conventionally used anti-IgE antibodies, but also binds to IgE very strongly due to having a binding capacity to IgE which is 70-fold higher than the conventionally used anti-IgE antibody, omalizumab, which allows an extended administration cycle. In addition, the polypeptide dimeric protein according to the present invention is a substance obtained by applying a modified Fc, which has IgE alone as a single target and does not bind to an Fc gamma receptor, and thus lacks antibody dependent cellular cytotoxicity (ADCC) and complement dependent cytotoxicity (CDC) functions. Therefore, unlike conventional anti-IgE antibodies containing an IgG1 Fc region, the polypeptide dimeric protein does not bind to an Fc gamma receptor, and thus can inhibit release of mediators caused by being bound to the Fc gamma receptor on the surface of mast cells, so that severe side effects such as occurrence of anaphylaxis which can be caused by binding between IgG1 and Fc gamma receptor III on mast cells can be minimized. Therefore, the polypeptide dimeric protein according to the present invention can be utilized as a new pharmaceutical composition which can replace therapeutic agents containing a conventional anti-IgE antibody.

### BRIEF DESCRIPTION OF DRAWINGS

FIGS. 1A-IC show results of SDS-PAGE and gel isoelectric focusing (IEF) for proteins produced in each cell line. Here, it can be seen that a truncated form is not generated at both reducing and non-reducing conditions, and that a content of acidic proteins is increased due to an increase in sialic acid content caused by introduction of a sialic acid transferase gene.

FIG. 2 illustrates SDS-PAGE results for non-reduced and reduced forms of the polypeptide dimeric protein according to an embodiment of the present invention. In particular, it can be seen that the polypeptide dimer has high purity even in culture supernatant which corresponds to Input.

FIG. 3 illustrates a graph showing a binding ability of omalizumab to IgE. The graph shows results obtained by immobilizing omalizumab and analyzing a binding ability thereof depending on IgE concentrations treated.

FIG. 4 illustrates a graph showing a binding ability, to IgE, of the polypeptide dimeric protein according to an embodiment of the present invention. The graph shows results obtained by immobilizing the dimeric protein and analyzing a binding ability thereof depending on IgE concentrations treated.

FIGS. 5A-5E illustrate results obtained by identifying interactions of the polypeptide dimeric protein (IgE_{TRAP}), an embodiment of the present invention, and omalizumab with IgG receptors FcγRI (FIG. 5A), FcγRIIA (FIG. 5B), FcγRIIB (FIG. 5C), FcγRIIIA (FIG. 5D), and FcγRIIIB (FIG. 5E) by bio-layer interferometry (BLI) assay.

FIG. 6 illustrates a graph obtained by quantifying a binding capacity between IgE_{TRAP} and IgG receptors, and between omalizumab and IgG receptors.

FIG. 7 illustrates a graph showing an inhibitory ability, on activity of mouse-derived mast cells, of the polypeptide dimeric protein (IgE_{TRAP}) according to an embodiment of the present invention depending on concentrations thereof.

FIG. 8 illustrates a graph showing a comparison between inhibitory abilities, on activity of human FcεRI-expressing mouse-derived mast cells, of the polypeptide dimeric protein (IgE_{TRAP}) according to an embodiment of the present invention and XOLAIR® (omalizumab) depending on concentrations thereof.

FIG. 9 illustrates a graph showing administration effects of the polypeptide dimeric protein according to an embodiment of the present invention in a food allergy model.

### DETAILED DESCRIPTION OF INVENTION

The present invention relates to a polypeptide dimer, comprising two monomers, each of which contains an extracellular domain (FcεRIa-ECD) of an alpha subunit of an IgE Fc receptor, in which the monomer contains a modified Fc region, and the modified Fc region and the FcεRIa-ECD are linked via a hinge of an IgD antibody.

As used herein, the term “IgE” means an antibody protein known as immunoglobulin E. IgE has an affinity to mast cells, blood basophils, or the like. In addition, reaction between an IgE antibody and an antigen (allergen) corresponding thereto causes an inflammatory reaction. In addition, IgE is known to be an antibody that causes anaphylaxis which occurs due to sudden secretion of mast cells or basophils.

As used herein, the term “IgE Fc receptor” is also referred to as Fcε receptor and binds to an Fc portion of IgE. There are two types for the receptor. The receptor having high affinity to IgE Fc is called Fcε receptor I (FcεRI). The receptor having low affinity to IgE Fc is called Fcε receptor II (FcεRII). FcεRI is expressed in mast cells and basophils. In a case where IgE antibodies bound to FcεRI are cross-linked by polyvalent antigens, degranulation occurs in mast cells or basophils, thereby releasing various chemical transmitter substances including histamine. This release leads to an immediate allergic reaction.

The FcεRI is a membrane protein composed of one α chain, one β chain, and two γ chains linked by a disulfide bond. Among these chains, a portion to which IgE binds is the α chain (FcεRIα). FcεRIα has a size of about 60 kDa, and is composed of a hydrophobic domain existing inside the cell membrane and a hydrophilic domain existing outside the cell membrane. In particular, IgE binds to an extracellular domain of the α chain.

Specifically, the alpha subunit of the IgE Fc receptor may have the amino acid sequence set forth in NP_001992.1. In addition, the extracellular domain (FcεRIa-ECD) of the alpha subunit of the IgE Fc receptor may have the amino acid sequence of SEQ ID NO: 1. In the present specification, the extracellular domain of the alpha subunit of the IgE Fc receptor may be a fragment or variant of the extracellular domain of the alpha subunit of the IgE Fc receptor, as long as the fragment or variant is capable of binding to IgE.

The variant may be prepared through a method of substituting, deleting, or adding one or more proteins in the wild-type FcεRIa-ECD (extracellular domain), as long as the method does not alter a function of the α chain of FcεRI. Such various proteins or peptides may be 90%, 91%, 92%, 93%, 94%, 95%, 96%, 97%, 98%, 99% or more identical to the amino acid sequence of SEQ ID NO: 1. In addition, the FcεRIa-ECD of SEQ ID NO: 1 may be encoded by a polynucleotide having the sequence of SEQ ID NO: 5.

In addition, as used herein, the term “modified Fc region” means a region in which a part of an Fc portion of an antibody has been modified. Here, the term “Fc region” refers to a protein which contains heavy chain constant region 2 (CH2) and heavy chain constant region 3 (CH3) of an immunoglobulin, and does not contain variable regions of heavy and light chains and light chain constant region 1 (CH1) of an immunoglobulin. In particular, the modified Fc region means a region obtained by substituting some amino acids in the Fc region or by combining different types of Fc regions. Specifically, the modified Fc region may have the amino acid sequence of SEQ ID NO: 2. In addition, the modified Fc region of SEQ ID NO: 2 may be encoded by a polynucleotide having the sequence of SEQ ID NO: 6.

In addition, the “modified Fc region” of the present invention may be in the form of having sugar chains in a native form, increased sugar chains relative to a native form, or decreased sugar chains relative to a native form, or may be in the form of being sugar chain-removed. Immunoglobulin Fc sugar chains may be modified by conventional methods such as chemical methods, enzymatic methods, and genetic engineering methods using microorganisms.

Here, the “modified Fc region” of the present invention may be a region that lacks antibody dependent cellular cytotoxicity (ADCC) and complement dependent cytotoxicity (CDC) functions due to having no binding site for FcγR or Clq. In addition, the modified Fc region and the FcεRIα-ECD may be linked via a hinge of an IgD antibody. The hinge of the IgD antibody is composed of 64 amino acids, and may selectively contain 20 to 60 consecutive amino acids, 25 to 50 consecutive amino acids, or 30 to 40 amino acids. In an embodiment, the hinge of the IgD antibody may be composed of 30 or 49 amino acids as shown below. In addition, the hinge of the IgD antibody may be a hinge variant obtained by modifying the hinge region, in which the hinge may contain at least one cysteine. Here, the hinge variant may be obtained by modifying some in a hinge sequence of the IgD antibody in order to minimize generation of truncated forms during a protein production process.

In an embodiment, the hinge may contain the following sequence: Arg Asn Thr Gly Arg Gly Gly Glu Glu Lys Lys Xaa1 Xaa2 Lys Glu Lys Glu Glu Gln Glu Glu Arg Glu Thr Lys Thr Pro Glu Cys Pro (SEQ ID NO: 17), where Xaa1 may be Lys or Gly, and Xaa2 may be Glu, Gly, or Ser. Specifically, the hinge may have the amino acid sequence of SEQ ID NO: 3 or SEQ ID NO: 19, thereby minimizing generation of truncated forms during a protein production process.

In another embodiment, the hinge may contain the following sequence: Ala Gln Pro Gln Ala Glu Gly Ser Leu Ala Lys Ala Thr Thr Ala Pro Ala Thr Thr Arg Asn Thr Gly Arg Gly Gly Glu Glu Lys Lys Xaa3 Xaa4 Lys Glu Lys Glu Glu Gln Glu Glu Arg Glu Thr Lys Thr Pro Glu Cys Pro (SEQ ID NO: 18), where Xaa3 may be Lys or Gly, and Xaa4 may be Glu, Gly, or Ser. Specifically, the hinge may have the amino acid sequence of SEQ ID NO: 4, thereby minimizing generation of truncated forms during a protein production process.

In particular, in the hinge having the sequence of SEQ ID NO: 4, at least one of Thr's may be glycosylated. Specifically, among the amino acids of SEQ ID NO: 18, the 13^{th}, 14^{th}, 18^{th}, or 19^{th} Thr's may be glycosylated. Preferably, all four amino acids may be glycosylated. Here, the glycosylation may be O-glycosylation.

In addition, as described above, the polypeptide dimer provided by the present invention may be in a form in which two monomers are bound to each other and each monomer is obtained by binding between an extracellular domain of an alpha subunit of an IgE Fc receptor and a modified Fc region. The polypeptide dimer may be in a form in which the same two monomers are bound to each other by cysteine located at a hinge site. In addition, the polypeptide dimer may be in a form in which two different monomers are bound to each other. For example, in a case where the two monomers are different from each other, the polypeptide dimer may be in a form in which one monomer contains the extracellular domain of the alpha subunit of the IgE Fc receptor, and the other monomer contains a fragment of the extracellular domain of the alpha subunit of the IgE Fc receptor. Here, an embodiment of the monomer may have the amino acid sequence of SEQ ID NO: 20, SEQ ID NO: 21, or SEQ ID NO: 22.

In addition, the polypeptide dimer provided by the present invention exhibits a binding capacity to IgE which is 10- to 100-fold, 20- to 90-fold, 20- to 70-fold, 30- to 70-fold, or 40- to 70-fold higher than omalizumab, an anti-IgE antibody, and may preferably exhibit a binding capacity to IgE which is 70-fold higher than omalizumab.

In yet another aspect of the invention, there is provided a polynucleotide encoding a monomer that contains an extracellular domain of an alpha subunit of an IgE Fc receptor to which a modified Fc region is bound.

Meanwhile, the polynucleotide may additionally contain a signal sequence or a leader sequence. As used herein, the term “signal sequence” means a nucleic acid encoding a signal peptide that directs secretion of a target protein. The signal peptide is translated and then cleaved in a host cell. Specifically, the signal sequence of the present invention is a nucleotide encoding an amino acid sequence that initiates protein translocation across the endoplasmic reticulum (ER) membrane. Useful signal sequences in the present invention include antibody light chain signal sequences, for example, antibody 14.18 (Gillies et al., J. Immunol. Meth 1989. 125:191-202), antibody heavy chain signal sequences, for example, the MOPC141 antibody heavy chain signal sequence (Sakano et al., Nature, 1980. 286:676-683), and other signal sequences known in the art (see, for example, Watson et al., Nucleic Acid Research, 1984. 12:5145-5164).

The signal sequences are well known in the art for their characteristics. The signal sequences typically contain 16 to 30 amino acid residues, and may contain greater or fewer amino acid residues. A typical signal peptide consists of three regions which are a basic N-terminal region, a central hydrophobic region, and a more polar C-terminal region. The central hydrophobic region contains 4 to 12 hydrophobic residues that immobilize the signal sequence through the membrane lipid bilayer during translocation of an immature polypeptide.

After initiation, a signal sequence is cleaved in the lumen of ER by cellular enzymes commonly known as signal peptidases. Here, the signal sequence may be a secretory signal sequence for tissue plasminogen activator (tPA), Herpes simplex virus glycoprotein D (HSV gD), or growth hormone. Preferably, secretory signal sequences used in higher eukaryotic cells, including mammals and the like, can be used. In addition, the secretory signal sequence may be substituted with a codon having a high frequency of expression in a host cell, and used.

Meanwhile, the extracellular domain monomer of the alpha subunit of the IgE Fc receptor to which a signal sequence and a modified Fc region are bound, may have the amino acid sequence of SEQ ID NO: 11 or SEQ ID NO: 13. The proteins of SEQ ID NO: 11 and SEQ ID NO: 13 may be encoded by polynucleotides having the sequences of SEQ ID NO: 12 and SEQ ID NO: 14, respectively.

In still yet another aspect of the present invention, there is provided an expression vector loaded with a polynucleotide encoding the monomer. Here, the polynucleotide may have the sequence of SEQ ID NO: 12 or SEQ ID NO: 14.

As used herein, the term “vector” is intended to be introduced into a host cell and to be capable of being recombined with a host cell genome and inserted thereinto. Alternatively, the vector is an episome and is understood as a nucleic acid unit containing a nucleotide sequence that can be autonomously replicated. The vectors include linear nucleic acids, plasmids, phagemids, cosmids, RNA vectors, viral vectors, and analogs thereof. Examples of the viral vectors include, but are not limited to, retroviruses, adenoviruses, and adeno-associated viruses. In addition, the plasmids may contain a selectable marker such as an antibiotic-resistant gene, and host cells harboring the plasmids may be cultured under selective conditions.

As used herein, the term “genetic expression” or “expression” of a target protein is understood to mean transcription of a DNA sequence, translation of an mRNA transcript, and secretion of a fusion protein product or a fragment thereof. A useful expression vector may be RcCMV (INVITROGEN™, Carlsbad) or a variant thereof. The expression vector may contain human cytomegalovirus (CMV) promoter for promoting continuous transcription of a target gene in mammalian cells, and a bovine growth hormone polyadenylation signal sequence for increasing a stability level of RNA after transcription.

In still yet another aspect of the present invention, there is provided a host cell into which the expression vector is introduced. As used herein, the term “host cell” refers to a prokaryotic or eukaryotic cell into which a recombinant expression vector can be introduced. As used herein, the terms “transduced”, “transformed”, and “transfected” means introducing a nucleic acid (for example, a vector) into a cell using techniques known in the art.

Preferred host cells that can be used in the present invention include immortal hybridoma cells, NS/0 myeloma cells, 293 cells, Chinese hamster ovary cells (CHO cells), HeLa cells, human amniotic fluid-derived cells (CapT cells), or COS cells. Preferably, the host cells may be CHO cells. On the other hand, the host cell may be one in which the vector and a vector loaded with a sialic acid transferase gene are introduced. Here, the sialic acid transferase may be 2,3-sialic acid transferase or 2,6-sialic acid transferase. Here, the 2,6-sialic acid transferase may have the amino acid sequence of SEQ ID NO: 15.

In still yet another aspect of the present invention, there is provided a pharmaceutical composition for treating or preventing an allergic disease, comprising the polypeptide dimer as an active ingredient.

In the present specification, the term “allergic disease” means a pathological symptom caused by an allergic reaction mediated by mast cell activation such as mast cell degranulation. Such allergic diseases include food allergy, atopic dermatitis, asthma, allergic rhinitis, allergic conjunctivitis, allergic dermatitis, allergic contact dermatitis, anaphylaxis, urticaria, pruritus, insect allergy, chronic idiopathic urticaria, drug allergy, and the like. In particular, the allergic diseases may be IgE-mediated.

In the composition for treating or preventing allergic disease of the present invention, an active ingredient may be contained in any amount (effective amount) depending on use, formulation, blending purpose, and the like, as long as the active ingredient can exhibit anti-allergic activity. A typical effective amount of the active ingredient will be determined within a range of 0.001% by weight to 20.0% by weight based on a total weight of the composition. Here, “effective amount” refers to an amount of an active ingredient which is capable of inducing an anti-allergic effect. Such an effective amount can be determined experimentally within the ordinary skill of those skilled in the art.

Here, the pharmaceutical composition may further contain a pharmaceutically acceptable carrier. For the pharmaceutically acceptable carrier, any carrier can be used as long as the carrier is a non-toxic substance suitable for delivery to a patient. Distilled water, alcohol, fat, wax, and an inert solid may be contained as carriers. Pharmaceutically acceptable adjuvants (buffers and dispersants) may also be contained in the pharmaceutically composition.

Specifically, the pharmaceutical composition of the present invention contains, in addition to an active ingredient, a pharmaceutically acceptable carrier, and may be made into an oral or parenteral formulation depending on a route of administration by a conventional method known in the art. Here, the term “pharmaceutically acceptable” means not having more toxicity than a subject to be applied (prescribed) can accommodate without inhibiting activity of the active ingredient.

In a case where the pharmaceutical composition of the present invention is made into an oral formulation, the pharmaceutical composition may be made into formulations such as powders, granules, tablets, pills, sugar coating tablets, capsules, liquids, gels, syrups, suspensions, and wafers, together with suitable carriers, in accordance with methods known in the art. Here, examples of suitable pharmaceutically acceptable carriers can include sugars such as lactose, glucose, sucrose, dextrose, sorbitol, mannitol, and xylitol, starches such as corn starch, potato starch, and wheat starch, celluloses such as cellulose, methylcellulose, ethylcellulose, sodium carboxymethyl cellulose, and hydroxypropylmethyl cellulose, polyvinyl pyrrolidone, water, methylhydroxybenzoate, propylhydroxybenzoate, magnesium stearate, mineral oil, malt, gelatin, talc, polyol, vegetable oil, and the like. In a case of being made into preparations, the preparations can be carried out, as necessary, by including diluents and/or excipients such as a filler, an extender, a binder, a wetting agent, a disintegrant, and a surfactant.

In a case where the pharmaceutical composition of the present invention is made into a parenteral formulation, the pharmaceutical composition may be made into preparations in the form of injections, transdermal drugs, nasal inhalers, and suppositories, together with suitable carriers, in accordance with methods known in the art. In a case of being prepared into injections, sterilized water, ethanol, polyol such as glycerol and propylene glycol, or a mixture thereof may be used as a suitable carrier. For the carrier, isotonic solutions such as Ringer's solution, phosphate buffered saline (PBS) containing triethanolamine, sterile water for injection, and 5% dextrose, and the like may be preferably used.

Preparation of the pharmaceutical composition is known in the art and specifically, reference can be made to Remington's Pharmaceutical Sciences (19th ed., 1995) and the like. The document is considered part of the present specification.

A preferable daily dosage of the pharmaceutical composition of the present invention is ranged from 0.01 ug/kg to 10 g/kg, and preferably from 0.01 mg/kg to 1 g/kg, depending on the patient's condition, body weight, sex, age, disease severity, or route of administration. Administration may be carried out once or several times a day. Such a dosage should in no way be interpreted as limiting the scope of the present invention.

The subject to which the composition of the present invention can be applied (prescribed) is a mammal and a human, with a human being particularly preferred. The composition for anti-allergy of the present invention may further comprise, in addition to the active ingredient, any compound or natural extract, on which safety has already been verified and which is known to have anti-allergic activity, for the purpose of raising and reinforcing the anti-allergic activity.

In another aspect of the present invention, there is provided a food composition for ameliorating and alleviating an allergic symptom, comprising the polypeptide dimer as an active ingredient.

Here, the polypeptide dimer may be bound to an appropriate delivery unit for efficient delivery into the intestines. The food composition of the present invention may be prepared in any form and may be, for example, prepared in the form of beverages such as tea, juice, carbonated beverage, and ionic beverage, processed dairy products such as milk and yogurt, health functional food preparations such as tablets, capsules, pills, granules, liquids, powders, flakes, pastes, syrups, gels, jellies, and bars, or the like. In addition, the food composition of the present invention may fall within any product category in legal or functional classification as long as the food composition complies with the enforcement regulations at the time of being manufactured and distributed. For example, the food composition may be a health functional food according to the Health Functional Foods Act, or may fall within confectioneries, beans, teas, beverages, special-purpose foods, or the like according to each food type in the Food Code of Food Sanitation Act (standards and specifications for food, notified by Food and Drug Administration). With regard to other food additives that may be contained in the food composition of the present invention, reference can be made to the Food Code or the Food Additive Code according to the Food Sanitation Act.

In yet another aspect of the present invention, there is provided a method for producing a polypeptide dimer, comprising a step of culturing a host cell into which a polynucleotide encoding a monomer and a sialic acid transferase gene have been introduced; and a step of recovering a polypeptide dimer.

Here, the polynucleotide encoding the monomer may be introduced into the host cell in the form of being loaded on an expression vector. In addition, the sialic acid transferase gene may be introduced into the host cell in the form of being loaded on a vector.

First, a step of introducing, into a host cell, a vector loaded with a polynucleotide encoding a monomer and a vector loaded with a sialic acid transferase gene is carried out. Here, the sialic acid transferase may be 2,3-sialic acid transferase or 2,6-sialic acid transferase.

Next, a step of culturing the transformed cell is carried out.

Finally, a step of recovering a polypeptide dimer is carried out. Here, the polypeptide dimer may be purified from a culture medium or a cell extract. For example, after obtaining supernatant of a culture medium in which the polypeptide dimer is secreted, the supernatant may be concentrated using a commercially available protein concentration filter, for example, an AMICON® or MILLIPORE® PELLICON® ultrafiltration unit. Then, the concentrate may be purified by methods known in the art. For example, the purification may be achieved using a matrix coupled to protein A.

In still yet another aspect of the present invention, there is provided a polypeptide dimer produced by the above-described method for producing a dimer.

Here, the polypeptide dimer has a high sialic acid content, and thus has a very high content of acidic proteins relative to the theoretical pI value.

In still yet another aspect of the present invention, there is provided a pharmaceutical composition for treating or preventing an allergic disease, comprising, as an active ingredient, the polypeptide dimer produced by the above-described method for producing a dimer.

In still yet another aspect of the present invention, there is provided a food composition for ameliorating or alleviating an allergic symptom comprising, as an active ingredient, the polypeptide dimer produced by the above-described method for producing a dimer.

In still yet another aspect of the present invention there is provided a method for treating or preventing an allergic disease, comprising a step of administering to a subject a polypeptide dimer that contains two monomers, each of which contains the extracellular domain (FcεRIa-ECD) of the alpha subunit of the IgE Fc receptor.

The subject may be a mammal, preferably a human. Here, administration may be achieved orally or parenterally. Here, parenteral administration may be performed by methods such as subcutaneous administration, intravenous administration, mucosal administration, and muscular administration.

Hereinafter, the present invention will be described in more detail with reference to the following examples. However, the following examples are intended to merely illustrate the present invention, and the scope of the present invention is not limited only thereto.

### Example 1. Preparation of Polypeptide Containing FcεRIα-ECD and Modified Fc Region

A C-terminal modified polypeptide of the extracellular domain (FcεRIα-ECD) of the alpha subunit of the IgE Fc receptor was prepared according to the method disclosed in U.S. Pat. No. 7,867,491.

First, in order to express a protein (FcεRIαECD-Fc1), a protein (FcεRIαECD-Fc2), and a protein (FcΣR1αECD-Fc3), in which the extracellular domain of the α-chain of FcεRI having the amino acid sequence of SEQ ID NO: 1 and the modified immunoglobulin Fc of SEQ ID NO: 2 are linked via a hinge of SEQ ID NO: 19, a hinge of SEQ ID NO: 3, and a hinge of SEQ ID NO: 4, respectively, cassettes obtained by linking the gene encoding each protein were cloned into the pAD15 vectors (Genexin, Inc.) to construct FcεRIαECD-Fc protein expression vectors. Then, each of the expression vectors was transduced into CHO DG44 cells (from Dr. Chasm, Columbia University, USA).

Here, at the time of being transduced into the cell line, an expression vector obtained by cloning an α-2,6-sialic acid transferase gene into the pCI Hygro vector (INVITROGEN™) was simultaneously transduced to separately prepare cell lines which are capable of expressing FcεRIαECD-Fc2ST and FcεRIα ECD-Fc3ST proteins to which sialic acid is added.

As a primary screening procedure, HT selection was carried out using 5-hydroxytryptamine (HT)-free 10% dFBS medium (GIBCO™, USA, 30067-334), MEMα medium (GIBCO™, 12561, USA, Cat No. 12561-049), and HT+ medium (GIBCO™, USA, 11067-030). Then, methotrexate (MTX) amplification was performed using HT-selected clones to amplify productivity using the dihydrofolate reductase (DHFR)-system.

After completion of the MTX amplification, subculture was carried out about 1 to 5 times for cell stabilization for the purpose of evaluation of productivity. Thereafter, unit productivity evaluation of the MTX-amplified cells was performed. The results are shown in Table 1 below.

TABLE 1

| | | | Productivity | | |
|---|---|---|---|---|---|
| | | MTX | 3-day culture | | Batch |
| | | concen- | | ug/10⁶ | culture |
| Version | Media | tration | ug/mL | cells | (mg/ml) |
| FcεRIαECD-Fc2 | Ex- | 500 nM | 37.23 | 20.9 | 225 |
| FcεRIαECD-Fc2 + | cellDHFR | 100 nM | 45.4 | 25.1 | 338.2 |
| a2,6-ST | | | | | |
| FcεRIαECD-Fc3 | | 2 uM | 27.0 | 16.9 | 180.4 |
| FcεRIαECD-Fc3 + | | 1 uM | 17.5 | 10.2 | 101.7 |
| a2,6-ST | | | | | |

As shown in Table 1, the FcεRIαECD-Fc3 cell line exhibited productivity of 16.9 ug/10⁶ cells after the methotrexate amplification at 2 uM. On the other hand, the FcεRIαECD-Fc3 cell line (FcεRIαECD-Fc3ST) co-transduced with 2,6-sialic acid transferase exhibited productivity of 10.2 ug/10⁶ cells after the methotrexate amplification at 1 uM. In addition, the FcεRIαECD-Fc2 cell line exhibited productivity of 20.9 ug/10⁶ cells under the methotrexate amplification condition at 0.5 uM. In addition, the FcεRIαECD-Fc2 cell line (FcεRIαECD-Fc2ST) co-transduced with 2,6-sialic acid transferase exhibited productivity of 25.1 ug/10⁶ cells after the methotrexate amplification at 0.1 uM. That is, it was identified that the FcεRIαECD-Fc2 cell line co-transfected with 2,6-sialic acid transferase, which had been selected under the methotrexate amplification condition at 0.1 uM, exhibits the most excellent productivity.

### Example 2. Purification of FcεRIα ECD Fusion Protein and Identification of Purity Thereof

Among the cell lines selected in Example 1 above, i) FcεRIαECD-Fc3, ii) FcεRIαECD-Fc3ST, and iii) FcεRIαECD-Fc2ST were cultured at a 60 ml scale by a batch culture method. The resulting cultures were purified using a Protein-A affinity column, and then purified proteins were subjected to SDS-PAGE and size-exclusion HPLC (SE-HPLC) to identify purity of the proteins.

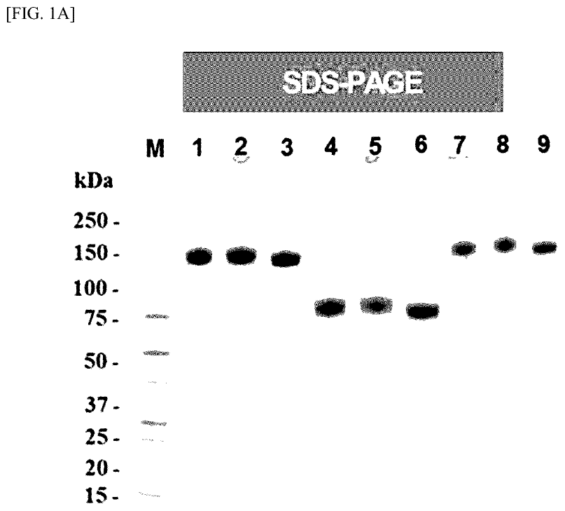

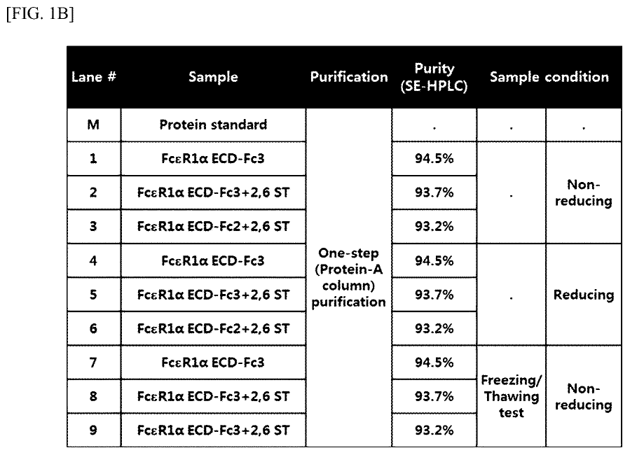

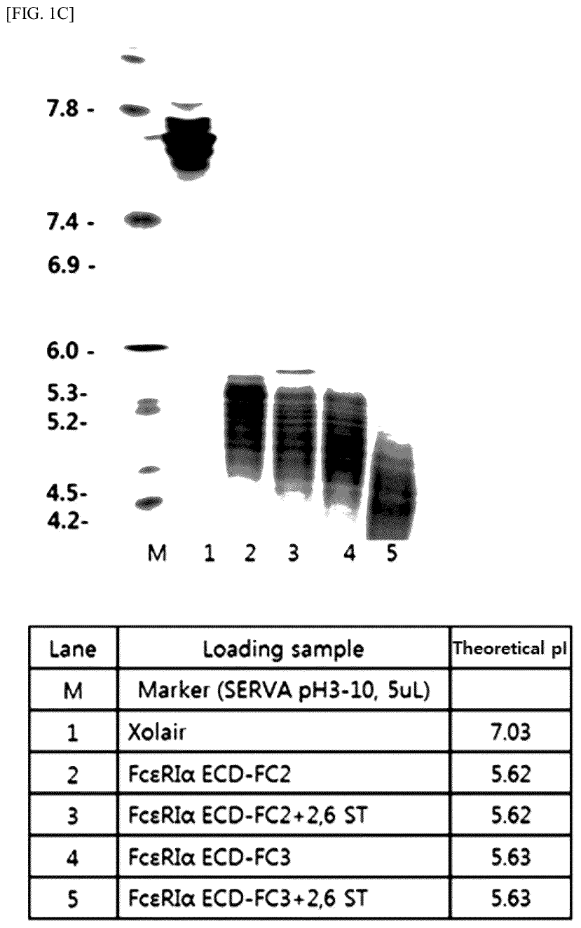

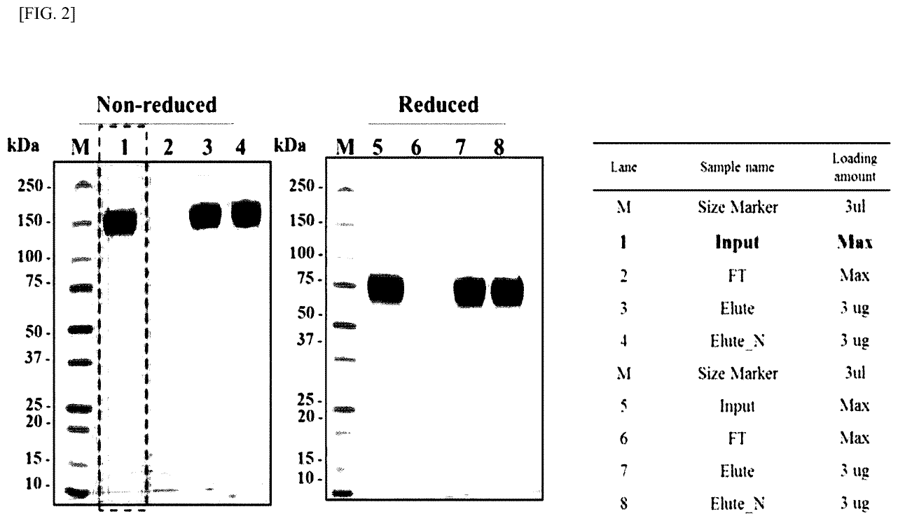

As shown in FIGS. 1A-1C, it was identified that all respective proteins purified by the SE-HPLC method have purity of 93% or higher. In addition, as a result of SDS-PAGE analysis, it was identified that proteins having sizes of about 150 kDa and about 75 kDa are detected, respectively, in the non-reducing and reducing conditions (FIG. TA, Lanes 1 to 6). From this, it was found that the Fc-bound FcεRIαECD forms a dimer. In addition, no impurities such as a truncated form were observed in the SDS-PAGE results. In particular, even after the process of thawing/freezing (FIG. TA, Lanes 7 to 8), it was identified that all proteins have purity of 93% or higher, and has no impurities. From this, it was found that production of truncated protein forms is decreased relative to the FcεRIαECD-Fc1 to which the wild type IgD hinge had been applied.

Here, Gel-IEF was performed under the following test conditions to identify a degree of sialic acid content in the proteins following introduction of the sialic acid transferase. From this, it was identified that a content of acidic proteins is increased due to increased sialic acid content.

TABLE 2
Test conditions

| | |
|---|---|
| Gel | pH3-10 IEF gel 1.0 mm |
| Sample buffer | IEF sample buffer (2×) |
| Loading condition | 100 V 1 hr, 200 V 1 hr, 500 V 2 hr |

In order to identify reproducibility of purification yield, the FcεRIαECD-Fc2ST cell line was batch-cultured in a 1 L flask at a 250 ml scale and purified using a Protein-A affinity column. Subsequently, the culture supernatant and the purified product were subjected to running on a 4% to 15% TGX™ Precast Protein gel (TGX: Tris-Glycine eXtended, Bio-Rad Laboratories, Inc.) for 30 minutes at a condition of Tris-Glycine SDS (TGS) buffer and 200 V, and then subjected to SDS PAGE analysis. As a result, it was identified that not only proteins with very high purity (98% or higher) are purified even by only the first step purification but also proteins are expressed with very high purity even in the culture supernatant. This indicates that process development steps can be simplified in developing the FcεRIαECD-Fc protein, which had been expressed in the cell line in question, into a medical product, and as a result, it is highly likely for the development cost of the medical product to be remarkably decreased.

### Example 3. Identification of Binding Ability of FcεRIα ECD Fusion Protein to IgE

A binding ability to IgE was comparatively measured for the four proteins, i) FcεRIαECD-Fc2, ii) FcεRIαECD-Fc2ST, iii) FcεRIαECD-Fc3, and iv) FcεRIαECD-Fc3ST which had been purified through the method of Example 2 above, and the commercially available anti-IgE antibody, omalizumab (trade name: XOLAIR®). Specifically, the binding ability to IgE was measured by coating IgE on the channel of the Protein GLC sensor chip (Bio-Rad Laboratories, Inc., Cat #176-5011), and causing omalizumab or each FcεR1αECD-Fc protein at various concentrations to flow at a rate of 30 μl per minute.

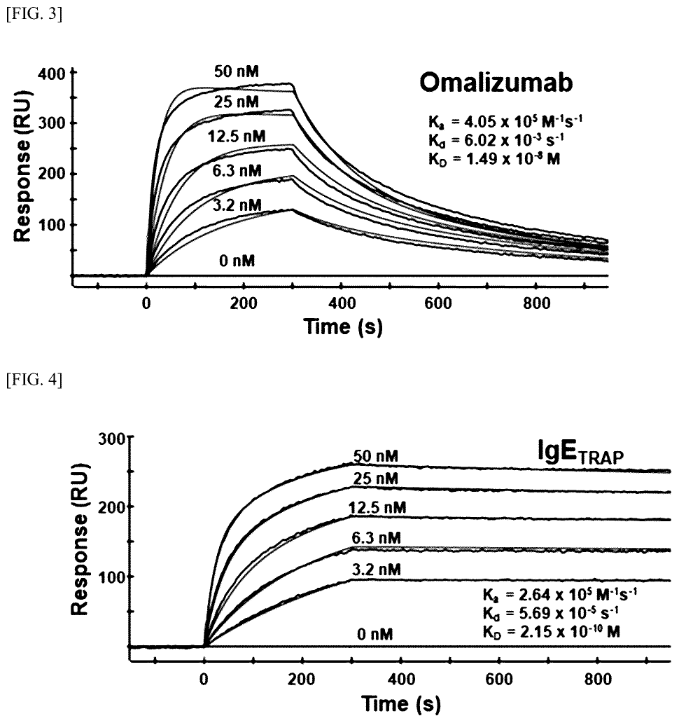

The experiments were conducted by identifying zero base using 25 mM NaOH as a regeneration buffer, and then repeating the above steps. Thereafter, a binding curve was identified using a protein binding analyzer (PROTEON™ XPR36 protein interaction array system, Bio-Rad Laboratories, Inc., USA). The results are shown in Table 3, and FIGS. 3 and 4.

TABLE 3

| Samples items | | FcεRIa ECD-Fc | | Omalizumab | |
|---|---|---|---|---|---|
| Drug type | | Fc fusion protein | | Anti-IgE Ab | Remarks |
| Binding | ka | Fc2 | 2.14 × 10⁵ | 4.05 × 10⁵ | 1.9-fold weaker than omalizumab |
| affinity | (association | Fc2ST | 2.64 × 10⁵ | | 1.5-fold weaker than omalizumab |
| | rate) | Fc3 | 1.98 × 10⁵ | | 2.0-fold weaker than omalizumab |
| | | Fc3ST | 2.40 × 10⁵ | | 1.7-fold weaker than omalizumab |
| | kd | Fc2 | 8.29 × 10⁻⁵ | 6.02 × 10⁻³ | 73-fold better than omalizumab |
| | (dissociation | Fc2ST | 5.69 × 10⁻⁵ | | 106-fold better than omalizumab |
| | rate) | Fc3 | 1.33 × 10⁻⁴ | | 45-fold better than omalizumab |
| | | Fc3ST | 1.49 × 10⁻⁴ | | 40-fold better than omalizumab |
| | KD (kd/ka) | Fc2 | 3.88 × 10⁻¹⁰ | 1.49 × 10⁻⁸ | 38-fold better than omalizumab |
| | | Fc2ST | 2.16 × 10⁻¹⁰ | | 69-fold better than omalizumab |
| | | Fc3 | 6.72 × 10⁻¹⁰ | | 22-fold better than omalizumab |
| | | Fc3ST | 6.21 × 10⁻¹⁰ | | 24-fold better than omalizumab |

As shown in Table 3, the association rate (ka) value of the polypeptide dimer according to an embodiment of the present invention was measured to be 1.5- to 2.0-fold lower than that of omalizumab. That is, it was found that a binding ability thereof to substances other than IgE is 1.5- to 2.0-fold lower than that of omalizumab. In addition, the dissociation rate (kd) value of the polypeptide dimer according to an embodiment of the present invention was measured to be 40- to 106-fold higher than that of omalizumab. In addition, as shown in FIGS. 3 and 4, it was able to identify that omalizumab loses its binding to IgE in a case where a certain period of time has passed after the binding, whereas once the polypeptide dimer of the FcεRIαECD fusion protein of the present invention binds to IgE, the polypeptide dimer is not separated from IgE.

That is, it can be seen that the polypeptide dimer of the present invention is not easily separated from IgE, and has a much better ability to maintain its bound state than omalizumab. As a result, it can be seen that the polypeptide dimer according to an embodiment of the present invention has an equilibrium dissociation constant (KD<kd/ka>) value which is 22- to 69-fold higher than omalizumab. From this, it was identified that the FcεRIαECD fusion protein of the present invention has a remarkably increased binding ability to IgE as compared with omalizumab. In particular, it was identified that the FcεRIαECD-Fc2 (FcεRIαECD-Fc2ST) to which sialic acid is added exhibits the highest IgE-binding capacity which is 69-fold higher than omalizumab.

### Example 4. Identification of Binding Ability of FcεRIα ECD Fusion Protein to IgG Receptor

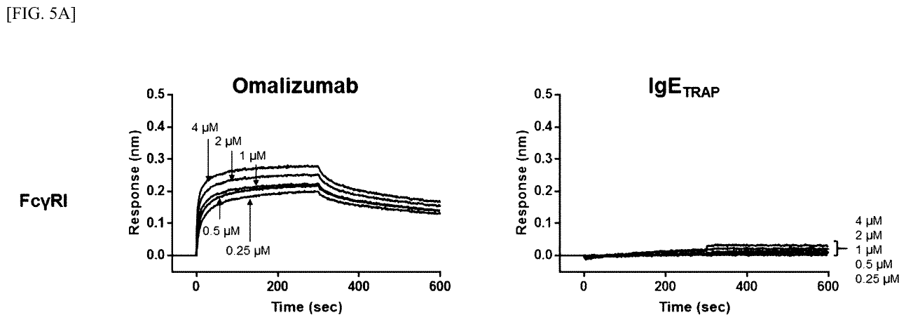

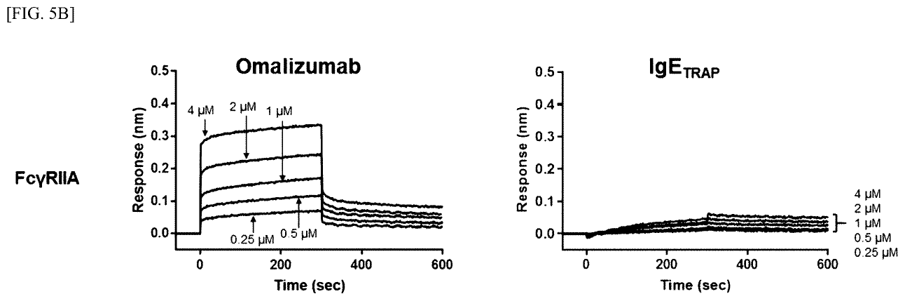

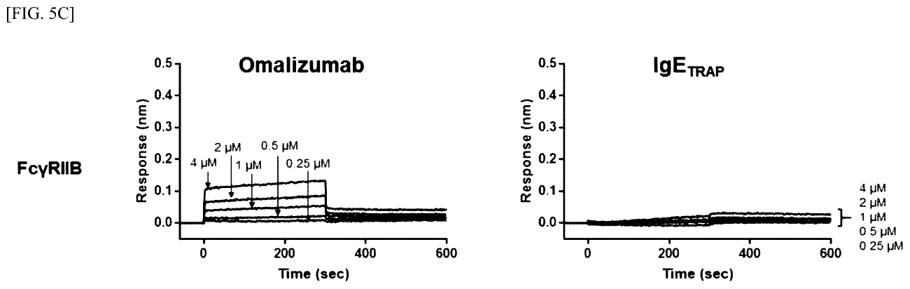

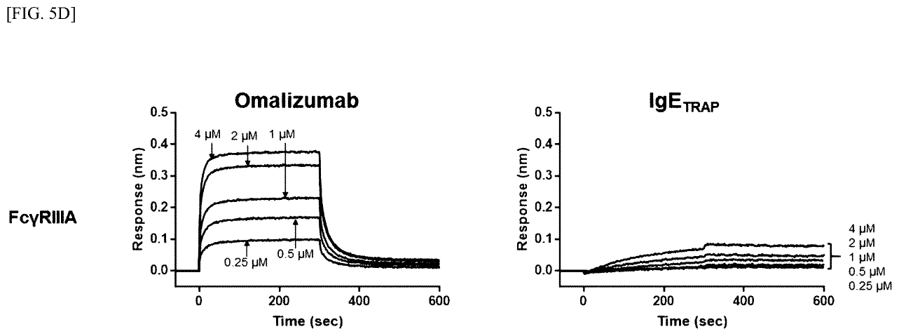

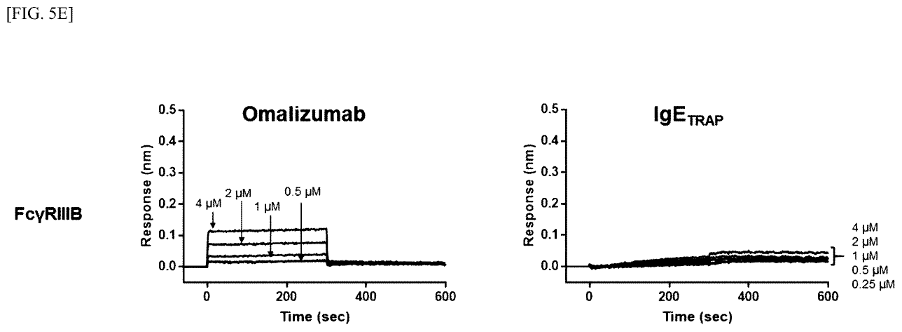

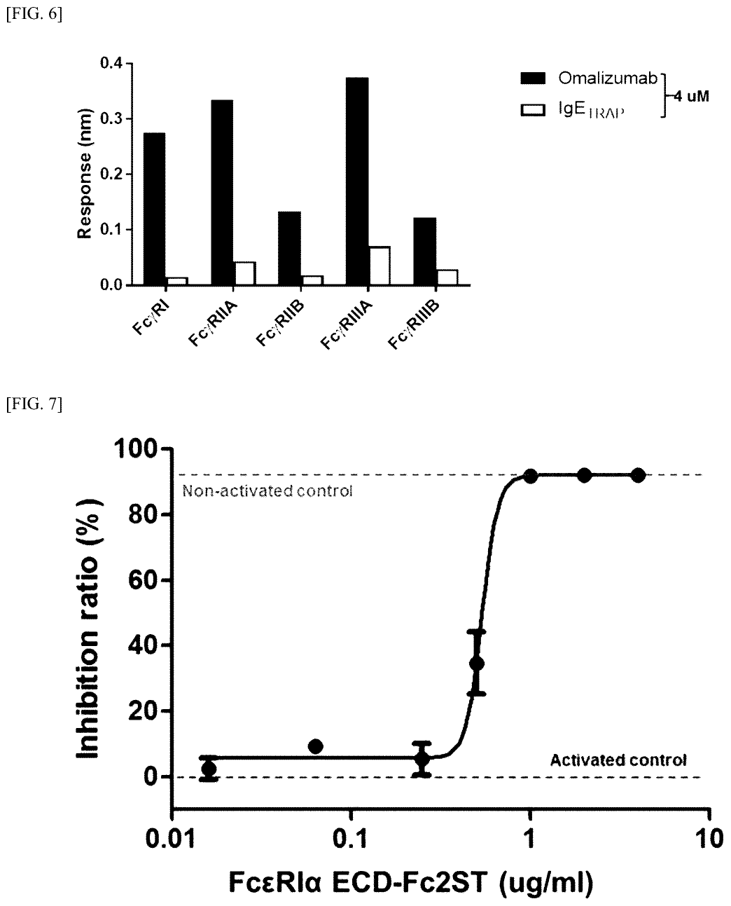

A degree of binding of IgE_{TRAP} and omalizumab to IgG receptors was identified using the OCTET® RED384 system (FORTEBIO™, Sartorius AG, CA, USA). FcγRI, FcγRIIA, FcγRIIB, FcγRIIIA, and FcγRIIIB recombinant proteins (R & D Systems Inc., 5 μg/ml) were immobilized in 300 mM acetate buffer (pH 5) on the activated AR2G biosensor. As a running buffer, PBS containing 0.1% Tween-20 and 1% bovine serum was used. All experiments were carried out at 30° C. with a sample plate shaker at a rate of 1,000 rpm. The results are shown in FIGS. 5A to 5E, and binding abilities of omalizumab and IgE_{TRAP} to the IgG receptors are quantified and shown in FIG. 6.

### Example 5. Identification of Activity of FcεRIα ECD Fusion Protein Through Beta-Hexosaminidase Assay in Mouse Bone Marrow-Derived Mast Cells

Beta-hexosaminidase assay was performed for in vitro activity analysis of the FcεRIαECD fusion protein of the present invention. Specifically, the FcεRIαECD-Fc2 protein according to an embodiment of the present invention was mixed, at each concentration, with mouse IgE (1 ug/mL), and incubated at room temperature (20° C.) for 30 minutes to prepare samples. Mouse bone marrow-derived mast cells in culture for mast cell activation were washed with Hank's balanced salt solution (HBSS) buffer to remove the medium, and the number of cells was measured. Then, an adjustment was made so that 5×10⁵ cells were injected into 40 μL of HBSS buffer.

Then, 50 μL of the sample solution prepared through the pre-incubation was added to the activated mast cells. Then, the resultant was incubated in a 5% CO₂ incubator at 37° C. for 30 minutes. Subsequently, after the addition of each 10 μL of DNP (2,4-dinitrophenol, 100 ng/mL), which is a foreign antigen, incubation was performed again at 37° C. for 30 minutes in 5% CO₂, and then 30 μL of the supernatant was separated. 30 μL of the separated supernatant and 30 μL of the substrate (4-nitrophenyl N-acetyl-β-D-glucosaminide, 5.84 mM) were mixed well, and then incubated at 37° C. for 20 minutes in 5% CO₂. Then, 140 μL of 0.1 M sodium carbonate buffer (pH 10) as a stop solution was added to terminate the reaction. Thereafter, absorbance at 405 nm was measured to identify a secretion amount of β-hexosaminidase secreted by the foreign antigen in the activated mast cells. The results are shown in FIG. 7.

As shown in FIG. 7, the polypeptide dimer of an embodiment of the present invention exhibited a mast cell inhibition ratio of about 49.4% in a case of having half (0.5 ug/mL) the concentration of mouse IgE, and exhibited a mast cell inhibition ratio of about 99.4% in a case of having the same concentration (1 ug/mL) of mouse IgE. That is, it can be seen that IgE-induced activity of bone marrow-derived mast cells is greatly suppressed by the FcεRIa-ECD polypeptide dimer of the present invention.

### Example 6. Comparison of Activity of FcεRIα ECD Fusion Protein and Anti-Human IgE Antibody Using β-Hexosaminidase Assay in Human FcεRI-Expressing Bone Marrow-Derived Mast Cells

β-Hexosaminidase assay was conducted to identify superiority of the FcεRIα ECD fusion protein relative to XOLAIR® through in vitro activity analysis. The respective drugs, FcεRIαECD-Fc2ST (IgE_{TR}AP) and XOLAIR®, were prepared at each concentration, and then mixed with human IgE (1 ug/mL). Then, incubation was performed at room temperature for 30 minutes. During pre-incubation of the drug, a human FcεRI gene was introduced, and mast cells derived from and differentiated from mouse bone marrow, in which the mouse FcεRI gene had been removed, were prepared. The prepared mast cells were washed with HBSS buffer, and then 5×10⁵ cells were injected into 60 μL of HBSS buffer. 20 μL of the pre-incubated sample was added to the prepared mast cells, and then incubated in a 5% CO₂ incubator at 37° C. for 30 minutes.

Subsequently, after 20 μL of anti-human IgE antibody (BioLegend, Inc., Cat No. 325502, 0.5 ug/mL) was added, and then the resultant was incubated again in 5% CO₂ incubator at 37° C. for 30 minutes. Subsequently, after centrifugation at 1,500 rpm at 4° C., 30 μL of the supernatant was separated. 30 μL of the separated supernatant and 30 μL of the substrate (4-nitrophenyl N-acetyl-β-glucosaminide, 5.84 mM) were mixed well, and then incubated in a 5% CO₂ incubator at 37° C. for 25 minutes. Then, 140 μL of 0.1 M sodium carbonate buffer (pH 10) was added to terminate the reaction.

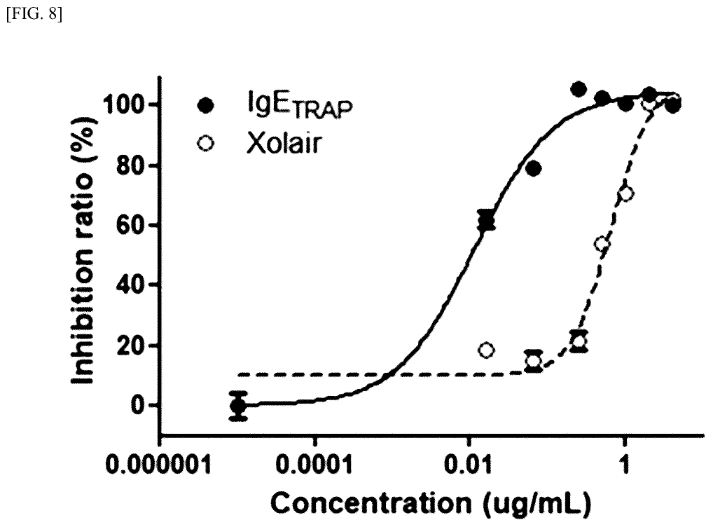

Subsequently, absorbance at 405 nm was measured to compare relative amounts of secreted β-hexosaminidase, and a mass cell-inhibitory effect depending on each drug concentration was identified. The results are shown in FIG. 8. As shown in FIG. 8, IC₅₀ of the FcεRIα ECD fusion protein was measured to be approximately 11.16 ng/mL, and IC₅₀₀f the XOLAIR® protein was measured to be approximately 649.8 ng/mL. Therefore, it was identified that the FcεRIα ECD fusion protein has a 58-fold higher inhibitory ability on mast cell activity than XOLAIR®.

### Example 7. In Vivo Assay of FcεRIα ECD Fusion Protein (Food Allergy Model)

50 μg of ovalbumin (OVA) and 1 mg of alum were intraperitoneally administered to Balb/c mice (Orientbio Inc.) two times at a 14-day interval to induce sensitization. Thereafter, 50 mg of OVA was orally administered five times in total on days 28, 30, 32, 34, and 36, to induce food allergy in intestines.

After the OVA was orally administered two times, that is, on day 31, the mice were divided into three groups, each containing 7 mice. The three divided groups were as follows: The first group receiving the FcεRIαECD-Fc2ST fusion protein at a high concentration (200 ug), the second group receiving the FcεRIαECD-Fc2ST fusion protein at a low concentration (20 ug), and the third group receiving nothing. While orally administering the OVA, it was identified whether diarrhea occurs due to food allergy induction. The mice were sacrificed on day 37, and the number of mast cells in the small intestine, the IgE concentration in blood, and the concentration of enzyme (mast cell protease-1 (MCPT-1)) with mast cell degranulation in blood were analyzed for the mice belonging to each group.

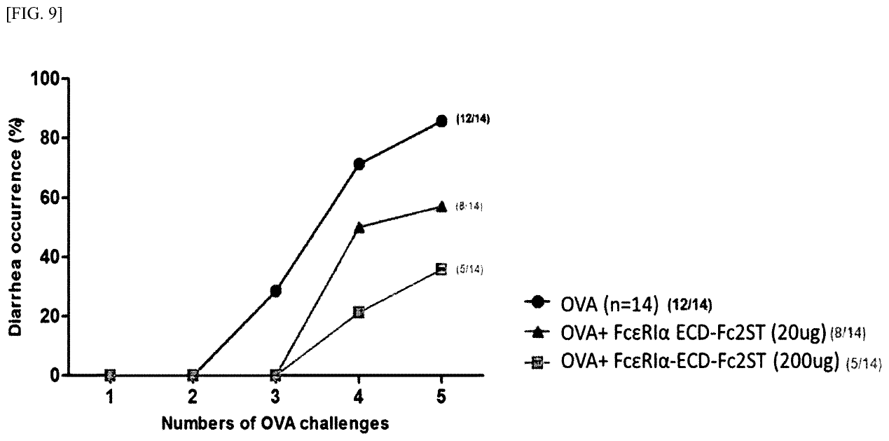

As shown in FIG. 9, it was identified that the mice belonging to the group receiving the FcεRIαECD-Fc2ST, which is a polypeptide dimer, at a high concentration exhibits an effect of alleviating food allergy in a concentration-dependent manner, as compared with the mice belonging to the group receiving nothing.

### [Sequence List Text]

SEQ ID NO: 1
VPQKPKVSLN PPWNRIFKGE NVTLTCNGNN FFEVSSTKWF HNGSLSEETN SSLNIVNAKF
EDSGEYKCQH QQVNESEPVY LEVFSDWLLL QASAEVVMEG QPLFLRCHGW RNWDVYKVIY
YKDGEALKYW YENHNISITN ATVEDSGTYY CTGKVWQLDY ESEPLNITVI KAPREKYWLQ
SEQ ID NO: 2
SHTQPLGVFL FPPKPKDTLM ISRTPEVTCV VVDVSQEDPE VQFNWYVDGV EVHNAKTKPR
EEQFNSTYRV VSVLTVLHQD WLNGKEYKCK VSNKGLPSSI EKTISKAKGQ PREPQVYTLP
PSQEEMTKNQ VSLTCLVKGF YPSDIAVEWE SNGQPENNYK TTPPVLDSDG SFFLYSRLTV
DKSRWQEGNV FSCSVMHEAL HNHYTQKSLS LSLGK
SEQ ID NO: 3
RNTGRGGEEK KGSKEKEEQE ERETKTPECP
SEQ ID NO: 4
AQPQAEGSLA KATTAPATTR NTGRGGEEKK GSKEKEEQEE RETKTPECP
SEQ ID NO: 5
gtgccccaga agcccaaggt gagcctgaac cctccctgga acagaatctt caagggcgag
aacgtgaccc tgacctgcaa cggcaacaac ttcttcgagg tgagcagcac caagtggttc
cacaatggca gcctgagcga ggagaccaac agctccctga acatcgtgaa cgccaagttc
gaggacagcg gcgagtacaa gtgccagcac cagcaggtga acgagagcga gcccgtgtac
ctggaggtgt tcagcgactg gctgctgctg caggccagcg ccgaggtggt gatggagggc
cagcccctgt tcctgagatg ccacggctgg agaaactggg acgtgtacaa ggtgatctac
tacaaggatg gcgaggccct gaagtactgg tacgagaacc acaacatctc catcaccaac
gccaccgtgg aggacagcgg cacctactac tgcacaggca aggtgtggca gctggactac
gagagcgagc ccctgaacat caccgtgatc aaggctccca gagagaagta ctggctgcag
SEQ ID NO: 6
tgcgtggtcg tggatgtgag ccaggaagat cccgaagtgc agttcaactg gtacgtggat
ggcgtggaag tgcacaacgc caagaccaag cccagagaag agcagttcaa ctccacctac
agagtggtga gcgtgctgac cgtgctgcac caggactggc tgaacggcaa ggagtacaag
tgcaaggtgt ccaacaaagg cctgcccagc tccatcgaga agaccatcag caaagccaaa
ggccagccca gagaacccca ggtgtacacc ctgcctccca gccaggaaga gatgaccaag
aaccaggtgt ccctgacctg cctggtgaaa ggcttctacc ccagcgacat cgccgtggag
tgggaaagca acggccagcc cgagaacaat tacaagacaa cccctcccgt gctggatagc
gatggcagct tctttctgta cagcagactg accgtggaca agagcagatg gcaggaaggc
aacgtgttca gctgcagcgt gatgcacgaa gccctgcaca accactacac ccagaagagc
ctgtccctga gcctgggcaa g
SEQ ID NO: 7
aggaacaccg gcagaggagg cgaggaaaag aaaggaagca aggagaagga ggagcaggag
gaaagagaaa ccaagacccc cgagtgcccc agccacaccc agcccctggg cgtgttcctg
ttccccccca agcccaagga caccctgatg atcagcagaa cccccgaggt gacc
SEQ ID NO: 8
gcccagcccc aggccgaggg cagcctggct aaggccacca cagctcccgc caccaccagg
aacaccggca gaggaggcga ggaaaagaaa ggaagcaagg agaaggagga gcaggaggaa
agagaaacca agacccccga gtgccccagc cacacccagc ccctgggcgt gttcctgttc
ccccccaagc ccaaggacac cctgatgatc agcagaaccc ccgaggtgac c
SEQ ID NO: 9
MDAMLRGLCC VLLLCGAVFV SPSHA
SEQ ID NO: 10
atggacgcca tgctgagagg cctgtgctgt gtgctgctgc tgtgcggcgc cgtgttcgtg
tcccctagcc acgcc
SEQ ID NO: 11
MDAMLRGLCC VLLLCGAVFV SPSHAVPQKP KVSLNPPWNR IFKGENVTLT CNGNNFFEVS
STKWFHNGSL SEETNSSLNI VNAKFEDSGE YKCQHQQVNE SEPVYLEVFS DWLLLQASAE
VVMEGQPLFL RCHGWRNWDV YKVIYYKDGE ALKYWYENHN ISITNATVED SGTYYCTGKV
WQLDYESEPL NITVIKAPRE KYWLQRNTGR GGEEKKGSKE KEEQEERETK TPECPSHTQP
LGVFLFPPKP KDTLMISRTP EVTCVVVDVS QEDPEVQFNW YVDGVEVHNA KTKPREEQFN
STYRVVSVLT VLHQDWLNGK EYKCKVSNKG LPSSIEKTIS KAKGQPREPQ VYTLPPSQEE
MTKNQVSLTC LVKGFYP SDIAVEWESNGQP ENNYKTTPPV LDSDGSFFLY SRLTVDKSRW
QEGNVFSCSV MHEALHNHYT QKSLSLSLGK
SEQ ID NO: 12
atggacgcca tgctgagagg cctgtgctgt gtgctgctgc tgtgcggcgc cgtgttcgtg
tcccctagcc acgccgtgcc ccagaagccc aaggtgagcc tgaaccctcc ctggaacaga
atcttcaagg gcgagaacgt gaccctgacc tgcaacggca acaacttctt cgaggtgagc
agcaccaagt ggttccacaa tggcagcctg agcgaggaga ccaacagctc cctgaacatc
gtgaacgcca agttcgagga cagcggcgag tacaagtgcc agcaccagca ggtgaacgag
agcgagcccg tgtacctgga ggtgttcagc gactggctgc tgctgcaggc cagcgccgag
gtggtgatgg agggccagcc cctgttcctg agatgccacg gctggagaaa ctgggacgtg
tacaaggtga tctactacaa ggatggcgag gccctgaagt actggtacga gaaccacaac
atctccatca ccaacgccac cgtggaggac agcggcacct actactgcac aggcaaggtg
tggcagctgg actacgagag cgagcccctg aacatcaccg tgatcaaggc tcccagagag
aagtactggc tgcagaggaa caccggcaga ggaggcgagg aaaagaaagg aagcaaggag
aaggaggagc aggaggaaag agaaaccaag acccccgagt gccccagcca cacccagccc
ctgggcgtgt tcctgttccc ccccaagccc aaggacaccc tgatgatcag cagaaccccc
gaggtgacct gcgtggtcgt ggatgtgagc caggaagatc ccgaagtgca gttcaactgg
tacgtggatg gcgtggaagt gcacaacgcc aagaccaagc ccagagaaga gcagttcaac
tccacctaca gagtggtgag cgtgctgacc gtgctgcacc aggactggct gaacggcaag
gagtacaagt gcaaggtgtc caacaaaggc ctgcccagct ccatcgagaa gaccatcagc
aaagccaaag gccagcccag agaaccccag gtgtacaccc tgcctcccag ccaggaagag
atgaccaaga accaggtgtc cctgacctgc ctggtgaaag gcttctaccc cagcgacatc
gccgtggagt gggaaagcaa cggccagccc gagaacaatt acaagacaac ccctcccgtg
ctggatagcg atggcagctt ctttctgtac agcagactga ccgtggacaa gagcagatgg
caggaaggca acgtgttcag ctgcagcgtg atgcacgaag ccctgcacaa ccactacacc
cagaagagcc tgtccctgag cctgggcaag
SEQ ID NO: 13
MDAMLRGLCC VLLLCGAVFV SPSHAVPQKP KVSLNPPWNR IFKGENVTLT CNGNNFFEVS
STKWFHNGSL SEETNSSLNI VNAKFEDSGE YKCQHQQVNE SEPVYLEVFS DWLLLQASAE
VVMEGQPLFL RCHGWRNWDV YKVIYYKDGE ALKYWYENHN ISITNATVED SGTYYCTGKV
WQLDYESEPL NITVIKAPRE KYWLQAQPQA EGSLAKATTA PATTRNTGRG GEEKKGSKEK
EEQEERETKT PECPSHTQPL GVFLFPPKPK DTLMISRTPE VTCVVVDVSQ EDPEVQFNWY
VDGVEVHNAK TKPREEQFNS TYRVVSVLTV LHQDWLNGKE YKCKVSNKGL PSSIEKTISK
AKGQPREPQV YTLPPSQEEM TKNQVSLTCL VKGFYPSDIA VEWESNGQPE NNYKTTPPVL
DSDGSFFLYS RLTVDKSRWQ EGNVFSCSVM HEALHNHYTQ KSLSLSLGK
SEQ ID NO: 14
atggacgcca tgctgagagg cctgtgctgt gtgctgctgc tgtgcggcgc cgtgttcgtg
tcccctagcc acgccgtgcc ccagaagccc aaggtgagcc tgaaccctcc ctggaacaga
atcttcaagg gcgagaacgt gaccctgacc tgcaacggca acaacttctt cgaggtgagc
agcaccaagt ggttccacaa tggcagcctg agcgaggaga ccaacagctc cctgaacatc
gtgaacgcca agttcgagga cagcggcgag tacaagtgcc agcaccagca ggtgaacgag
agcgagcccg tgtacctgga ggtgttcagc gactggctgc tgctgcaggc cagcgccgag
gtggtgatgg agggccagcc cctgttcctg agatgccacg gctggagaaa ctgggacgtg
tacaaggtga tctactacaa ggatggcgag gccctgaagt actggtacga gaaccacaac
atctccatca ccaacgccac cgtggaggac agcggcacct actactgcac aggcaaggtg
tggcagctgg actacgagag cgagcccctg aacatcaccg tgatcaaggc tcccagagag
aagtactggc tgcaggccca gccccaggcc gagggcagcc tggctaaggc caccacagct
cccgccacca ccaggaacac cggcagagga ggcgaggaaa agaaaggaag caaggagaag
gaggagcagg aggaaagaga aaccaagacc cccgagtgcc ccagccacac ccagcccctg
ggcgtgttcc tgttcccccc caagcccaag gacaccctga tgatcagcag aacccccgag
gtgacctgcg tggtcgtgga tgtgagccag gaagatcccg aagtgcagtt caactggtac
gtggatggcg tggaagtgca caacgccaag accaagccca gagaagagca gttcaactcc
acctacagag tggtgagcgt gctgaccgtg ctgcaccagg actggctgaa cggcaaggag
tacaagtgca aggtgtccaa caaaggcctg cccagctcca tcgagaagac catcagcaaa
gccaaaggcc agcccagaga accccaggtg tacaccctgc ctcccagcca ggaagagatg
accaagaacc aggtgtccct gacctgcctg gtgaaaggct tctaccccag cgacatcgcc
gtggagtggg aaagcaacgg ccagcccgag aacaattaca agacaacccc tcccgtgctg
gatagcgatg gcagcttctt tctgtacagc agactgaccg tggacaagag cagatggcag
gaaggcaacg tgttcagctg cagcgtgatg cacgaagccc tgcacaacca ctacacccag
aagagcctgt ccctgagcct gggcaag
SEQ ID NO: 15
MIHTNLKKKF SCCVLVFLLF AVICVWKEKK KGSYYDSFKL QTKEFQVLKS LGKLAMGSDS
QSVSSSSTQD PHRGRQTLGS LRGLAKAKPE ASFQVWNKDS SSKNLIPRLQ KIWKNYLSMN
KYKVSYKGPG PGIKFSAEAL RCHLRDHVNV SMVEVTDFPF NTSEWEGYLP KESIRTKAGP
WGRCAVVSSA GSLKSSQLGR EIDDHDAVLR FNGAPTANFQ QDVGTKTTIR LMNSQLVTTE
KRFLKDSLYN EGILIVWDPS VYHSDIPKWY QNPDYNFFNN YKTYRKLHPN QPFYILKPQM
PWELWDILQE ISPEEIQPNP PSSGMLGIII MMTLCDQVDI YEFLPSKRKT DVCYYYQKFF
DSACTMGAYH PLLYEKNLVK HLNQGTDEDI YLLGKATLPG FRTIHC
SEQ ID NO: 16
atgatccaca ccaacctgaa gaagaagttc agctgctgcg tgctggtgtt cctgctgttc
gccgtgatct gcgtgtggaa ggagaagaag aaaggcagct actacgacag cttcaagctg
cagaccaagg agttccaggt gctgaagagc ctgggcaagc tggccatggg cagcgacagc
cagagcgtgt ccagctcctc cacccaggat ccccacagag gcagacagac cctgggcagc
ctgagaggcc tggccaaggc caagcccgag gccagcttcc aggtgtggaa caaggacagc
agcagcaaga acctgatccc cagactgcag aagatctgga agaactacct gagcatgaac
aagtacaagg tgagctacaa aggacccgga cccggcatca agttcagcgc cgaggccctg
aggtgccacc tgagagacca cgtgaacgtg agcatggtgg aagtgaccga cttccccttc
aacaccagcg agtgggaagg ctacctgccc aaggagagca tcaggaccaa ggctggcccc
tggggcagat gcgccgtggt gagcagcgct ggcagcctga agagctccca gctgggcaga
gagatcgacg accacgatgc cgtgctgagg ttcaatggcg ctcccaccgc caacttccag
caggacgtgg gcaccaagac cacaatccgg ctgatgaaca gccagctggt gacaaccgag
aagcggttcc tgaaggacag cctgtacaac gagggcatcc tgatcgtgtg ggatcccagc
gtgtaccaca gcgacatccc caagtggtac cagaatcccg actacaactt cttcaacaac
tacaagacct atagaaagct gcaccccaac cagcccttct acatcctgaa gccccagatg
ccctgggagc tgtgggacat cctgcaggag atcagccctg aagagatcca gcccaaccct
ccctccagcg gcatgctggg cattatcatc atgatgaccc tgtgcgacca ggtggacatc
tacgagttcc tgcccagcaa gagaaagacc gacgtgtgct actactatca gaagttcttc
gacagcgcct gcaccatggg cgcctaccac cccctgctgt acgagaagaa cctggtgaag
cacctgaacc agggcaccga cgaggacatc tacctgctgg gcaaagccac cctgcccggc
ttcagaacca tccactgc
SEQ ID NO: 17
RNTGRGGEEK KXXKEKEEQE ERETKTPECP
SEQ ID NO: 18
AQPQAEGSLA KATTAPATTR NTGRGGEEKK XXKEKEEQEE RETKTPECP
SEQ ID NO: 19
RNTGRGGEEK KKEKEKEEQE ERETKTPECP
SEQ ID NO: 20
VPQKPKVSLN PPWNRIFKGE NVTLTCNGNN FFEVSSTKWF HNGSLSEETN SSLNIVNAKF
EDSGEYKCQH QQVNESEPVY LEVFSDWLLL QASAEVVMEG QPLFLRCHGW RNWDVYKVIY
YKDGEALKYW YENHNISITN ATVEDSGTYY CTGKVWQLDY ESEPLNITVI KAPREKYWLQ
RNTGRGGEEK KKEKEKEEQE ERETKTPECP SHTQPLGVFL FPPKPKDTLM ISRTPEVTCV
VVDVSQEDPE VQFNWYVDGV EVHNAKTKPR EEQFNSTYRV VSVLTVLHQD WLNGKEYKCK
VSNKGLPSSI EKTISKAKGQ PREPQVYTLP PSQEEMTKNQ VSLTCLVKGF YPSDIAVEWE
SNGQPENNYK TTPPVLDSDG SFFLYSRLTV DKSRWQEGNV FSCSVMHEAL HNHYTQKSLS
LSLGK
SEQ ID NO: 21
VPQKPKVSLN PPWNRIFKGE NVTLTCNGNN FFEVSSTKWF HNGSLSEETN SSLNIVNAKF
EDSGEYKCQH QQVNESEPVY LEVFSDWLLL QASAEVVMEG QPLFLRCHGW RNWDVYKVIY
YKDGEALKYW YENHNISITN ATVEDSGTYY CTGKVWQLDY ESEPLNITVI KAPREKYWLQ
RNTGRGGEEK KGSKEKEEQE ERETKTPECP SHTQPLGVFL FPPKPKDTLM ISRTPEVTCV
VVDVSQEDPE VQFNWYVDGV EVHNAKTKPR EEQFNSTYRV VSVLTVLHQD WLNGKEYKCK
VSNKGLPSSI EKTISKAKGQ PREPQVYTLP PSQEEMTKNQ VSLTCLVKGF YPSDIAVEWE
SNGQPENNYK TTPPVLDSDG SFFLYSRLTV DKSRWQEGNV FSCSVMHEAL HNHYTQKSLS
LSLGK
SEQ ID NO: 22
VPQKPKVSLN PPWNRIFKGE NVTLTCNGNN FFEVSSTKWF HNGSLSEETN SSLNIVNAKF
EDSGEYKCQH QQVNESEPVY LEVFSDWLLL QASAEVVMEG QPLFLRCHGW RNWDVYKVIY
YKDGEALKYW YENHNISITN ATVEDSGTYY CTGKVWQLDY ESEPLNITVI KAPREKYWLQ
AQPQAEGSLA KATTAPATTR NTGRGGEEKK GSKEKEEQEE RETKTPECPS HTQPLGVFLF
PPKPKDTLMI SRTPEVTCVV VDVSQEDPEV QFNWYVDGVE VHNAKTKPRE EQFNSTYRVV
SVLTVLHQDW LNGKEYKCKV SNKGLPSSIE KTISKAKGQP REPQVYTLPP SQEEMTKNQV
SLTCLVKGFY PSDIAVEWES NGQPENNYKT TPPVLDSDGS FFLYSRLTVD KSRWQEGNVF
SCSVMHEALH NHYTQKSLSL SLGK

1. A method for treating or preventing an allergic disease, comprising a step of administering to a subject the polypeptide dimer, which comprising:
two monomers, each of which comprises an extracellular domain of an alpha subunit of an IgE Fc receptor (FcεRIa-ECD), a modified Fc region, and a hinge,
wherein the modified Fc region and the FcεRIa-ECD are linked via the hinge.

2. The method of claim 1,
wherein the extracellular domain of the alpha subunit of the IgE Fc receptor is the amino acid sequence of SEQ ID NO: 1 or a fragment thereof.

3. The method of claim 1,
wherein the modified Fc region is SEQ ID NO: 2.

4. The method of claim 1,
wherein the hinge is a hinge region derived from an immunoglobulin IgD or a variant thereof.

5. The method of claim 4,
wherein the hinge region derived from an immunoglobulin IgD or the variant thereof contains at least one cysteine.

6. The method of claim 4,
wherein the hinge region derived from an immunoglobulin IgD or the variant thereof is Arg Asn Thr Gly Arg Gly Gly Glu Glu Lys Lys Xaa1 Xaa2 Lys Glu Lys Glu Glu Gln Glu Glu Arg Glu Thr Lys Thr Pro Glu Cys Pro (SEQ ID NO: 17),
Xaa1 is Lys or Gly, and
Xaa2 is Glu, Gly, or Ser.

7. The method of claim 4,
wherein the hinge region derived from an immunoglobulin IgD or the variant thereof is Ala Gln Pro Gln Ala Glu Gly Ser Leu Ala Lys Ala Thr Thr Ala Pro Ala Thr Thr Arg Asn Thr Gly Arg Gly Gly Glu Glu Lys Lys Xaa3 Xaa4 Lys Glu Lys Glu Glu Gln Glu Glu Arg Glu Thr Lys Thr Pro Glu Cys Pro (SEQ ID NO: 18),
Xaa3 is Lys or Gly, and
Xaa4 is Glu, Gly, or Ser.

8. The method of claim 1,
wherein the hinge has any one amino acid sequence selected from the group consisting of SEQ ID NO: 3, SEQ ID NO: 4, and SEQ ID NO: 19.

9. The method of claim 1,
wherein the allergic disease is any one selected from the group consisting of atopic dermatitis, asthma, allergic rhinitis, allergic conjunctivitis, allergic dermatitis, chronic idiopathic urticaria, and allergic contact dermatitis.
